(19) United States
(12) Patent Application Publication
(10) Pub. No.: US 2002/0033456 A1
(43) Pub. Date: Mar. 21, 2002

(54) Charged-particle beam irradiator and therapy system employing the same

(76) Inventors: Toshiki Tachikawa, Niihama-shi (JP); Kenji Sawada, Niihama-shi (JP)

Correspondence Address: ARENT FOX KINTNER PLOTKIN & KAHN, 1050 CONNECTICUT AVENUE, N.W., SUITE 600, WASHINGTON, DC, 20036, US

(21) Appl. No.: 09/904,105

(22) Filed: Jul. 13, 2001

(30) Foreign Application Priority Data
Jul. 13, 2000 (JP) 2000-212445

(51) Int. Cl. H01J 3/32; A61N 5/10

(52) U.S. Cl. 250/398; 250/492.3

(57) ABSTRACT

A charged-particle beam irradiator is provided with a plurality of scan electromagnets and configured such that kicks provided by the plurality of scan electromagnets are superimposed to form a collimated irradiation field at an exit of a final deflection electromagnet. A collimated irradiation field is thereby implemented by scanning irradiation even with a high magnetic field being provided by the final deflection electromagnet.

### BACKGROUND OF THE INVENTION

1. Field of the Invention

The present invention relates to a charged-particle beam irradiator which is adapted to use a scan electromagnet provided on an entrance side of a final deflection electromagnet in order to scan a charged-particle beam and thereby expand a field of irradiation, and to a therapy system that employs the irradiator.

2. Description of the Related Art

Commonly known is a proton beam therapy system, shown in FIG. 1, for use in the treatment of cancer. For example, the system comprises a therapy system 10, an accessory system 42, and a facility 44. The therapy system 10 comprises a proton beam accelerator 12, a beam transport system (BTS) 14, a rotatable irradiator (gantry) 30, and a stationary irradiator 40 for directing a proton beam in a fixed direction. The proton beam accelerator 12 has a cyclotron for accelerating protons to a predetermined energy level, and an Energy Selection System (ESS) for controlling the energy of the proton beam delivered from the cyclotron while restricting the dispersion of the energy as required. The beam transport system 14 stabilizes the track of the proton beam delivered from the proton beam accelerator 12 to transport the beam to an irradiation chamber without any loss. The rotary gantry 30 shapes the proton beam transported through the beam transport system 14 to variably direct the proton beam to accurately irradiate an affected part such as a cancerous tumor in a body of a patient. The accessory system 42 includes a diagnostic device for planning radiation treatment, a treatment planning system, and a treatment-apparatus machine tool. The facility 44 includes such as various types of power supplies mainly composed of a direct-current supply for supplying power to the accelerator, the beam transport system and the like, and a deionized water supply for supplying deionized water to directly cool current conductors (coils).

The rotary gantry 30 irradiates a patient at a given angle with a proton beam 33. For example, as shown in FIG. 2, the rotary gantry 30 comprises an irradiation nozzle 32 for fulfilling the requirements for irradiation such as the irradiation field and the irradiation depth, the end portion of the BTS 14 (not shown) for transporting the beam to the entrance of the irradiation nozzle 32, and a structure 36, provided at the end portion of the BTS 14, for rotating the irradiation nozzle 32. Adjacent to the irradiation nozzle 32, provided is a treatment bed 34 that includes a device for locating the affected part of the patient.

Upon providing treatment using such a therapy system, a proton beam having a narrow diameter has to be expanded to a square about 30 cm per side at a maximum according to the size of the affected part.

However, as shown in FIG. 3, for a gantry that employs the scattering method, the proton beam that is focused by a quadrupole electromagnet (Q magnet) 16 and deflected by a deflection electromagnet 18 and a final deflection electromagnet 20 is scattered by a scatterer 22 provided on an exit side of the final deflection electromagnet 20, being expanded within the distance from the scatterer 22 to an irradiation portion 35 (i.e., the treatment bed 34). Therefore, it is necessary to provide enough linear distance L to allow the proton beam to expand, thereby determining the diameter of the gantry 30. Thus, a diameter of 10 m is required of the gantry for delivering the proton beam from all directions within 360-degrees. Therefore, the cost of the system and the building increases, resulting in an impediment to the widespread use of the proton beam therapy system.

As means for eliminating this impediment, a scanning method is available for scanning the proton beam by a scan electromagnet 24 provided before the final deflection electromagnet 20, as shown in FIG. 4. In this scanning method, the proton beam is scanned by means of the scan electromagnet 24 disposed before the final deflection electromagnet 20. This allows the distance all the way from an entrance of the final deflection electromagnet 20 to the irradiation portion 35 to be used to expand the proton beam, thereby making it possible to significantly reduce the diameter of the gantry 30.

However, as shown in FIG. 5, the presence of a beam deflection provided by the final deflection electromagnet 20 causes a downstream convergence point to lie on a straight line connecting between the curvature center of the deflection electromagnet 20 and an upstream scan position. This causes the proton beam to tend to converge as shown in FIG. 6 or diverge as shown in FIG. 7 at the irradiation portion 35 unless the scan point is positioned at the entrance of the final deflection electromagnet 20. This has raised a problem that the irradiation area varies depending on the depth of irradiation and therefore made it impossible to provide a collimated irradiation field, the irradiation zone of which does not depend on the depth of irradiation, which is extremely important in irradiating an affected part.

In this regard, the shape of the final deflection electromagnet has been conventionally provided with an additional special contrivance such as a facial angle or a magnetic field gradient. For example, this is described in M. M. Kats, “Study of Gantry Optics for Proton and carbon ion Beams”, Proceedings of the 6th European Particle Accelerator Conference EPAC-98, Stockholm, Sweden, Jun. 22-26, 1998, p.2365 or E. Pedroni and H. Enge, “Beam optics design of compact gantry for proton therapy”, Medical & Biological Engineering & Computing, vol. 33, 1995, p.271-277.

These methods are effective when the deflection electromagnet has a magnetic field of 1.7 T to 1.8 T or less. Suppose a higher magnetic field of the order of 2 T is provided to thereby reduce the size of the gantry. This causes the magnetic poles to become saturated and thus has raised a problem that the distribution of the magnetic field cannot be easily controlled by the shape of the magnetic poles.

Further, M. Pavlovic, “Beam-optics study of the gantry beam delivery system for light-ion cancer therapy”, GSI Darmstadt, December 1995, p15-16 suggests using plurality of scan electromagnets. However, a way to decide positions of the scan electromagnets is not described.

### SUMMARY OF THE INVENTION

The present invention has been developed to solve the aforementioned prior art problems. It is therefore a first object of the present invention to provide a collimated irradiation field which maintains an irradiation zone at a constant area irrespective of a depth of irradiation upon performing scan irradiation even with a high magnetic field being provided by a final deflection electromagnet.

Furthermore, it is a second object of the present invention to further reduce the size of the rotary gantry employing the scanning method.

The present invention has accomplished the first object by providing a charged-particle beam irradiator that allows a scan electromagnet provided on an entrance side of a final deflection electromagnet to scan a charged-particle beam to expand an irradiation field. The irradiator includes a plurality of the scan electromagnets and kicks provided by the plurality of the scan electromagnets are superimposed to form a collimated irradiation field at an exit of the final deflection electromagnet.

Further, the plurality of the scan electromagnets can be arranged according to following equation.

a₁₁(s₁)·X₁′+a₁₁(s₂)·X₂′+ . . . +a₁₁(sₙ)·Xₙ′=0

where,

n: number of the electromagnets.

s₁ . . . sₙ : distance from each electromagnet to beam irradiated position

a₁₁(s): coefficient of beam transport matrix

X′: beam divergence (=kick) at the beam irradiated position

Further, the charged-particle beam irradiator can be adapted such that the plurality of scan electromagnets are interposed between the final deflection electromagnet and a deflection electromagnet disposed on an entrance thereof.

Alternatively, the charged-particle beam irradiator can also be adapted such that the plurality of scan electromagnets are disposed upstream from the deflection electromagnet at an entrance thereof.

Further, the charged-particle beam irradiator can be adapted such that the plurality of scan electromagnets, are disposed independent of each other in X and Y directions.

Furthermore, the present invention has accomplished the second object by allowing an affected part to be irradiated with a charged-particle beam using the aforementioned charged-particle beam irradiator.

The present invention makes it possible to implement a collimated irradiation field at an irradiation position without providing a special shape to the magnetic poles of the final deflection electromagnet. The present invention is extremely effective in providing a high magnetic field of about 2 T to the final deflection electromagnet in order to further reduce the gantry in size.

### BRIEF DESCRIPTION OF THE DRAWINGS

The preferred embodiments will be described with reference to the drawings, wherein like embodiments have been noted throughout the figures with like reference numerals.

FIG. 1 is a block diagram of an example of a conventional proton beam therapy system to which the present invention is to be applied, illustrating the overall configuration of the conventional proton beam therapy system;

FIG. 2 is a perspective view illustrating an example of a conventional rotary gantry of the aforementioned proton beam therapy system;

FIG. 3 is a view of an optical path showing an example of expanding the irradiation field of the conventional rotary gantry by employing the scattering method;

FIG. 4 is a view of an optical path showing an example of expanding the irradiation field by employing a conventional scanning method;

FIG. 5 is a diagram illustrating an example of a conventional relationship between scanning position and the convergence point in the scanning method;

FIG. 6 is a view of an optical path showing the state in which the beams tend to converge in the prior art scanning method;

FIG. 7 is a view of an optical path showing the state in which the beams tend to diverge in the prior art scanning method;

FIG. 8 is a view illustrating the configuration of an embodiment of a proton beam irradiation apparatus according to the present invention;

FIG. 9 is a view of an optical path showing an example of a collimated irradiation field that is implemented by the aforementioned embodiment;

FIG. 10 is a view showing kick by the single scan electromagnet.

FIG. 11 is a view showing combinations of kicks by two scan electromagnets, and

FIG. 12 is a view showing function of phase controller.

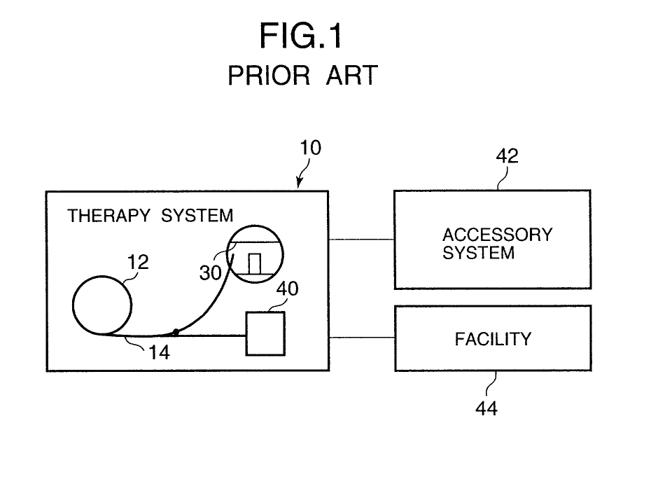

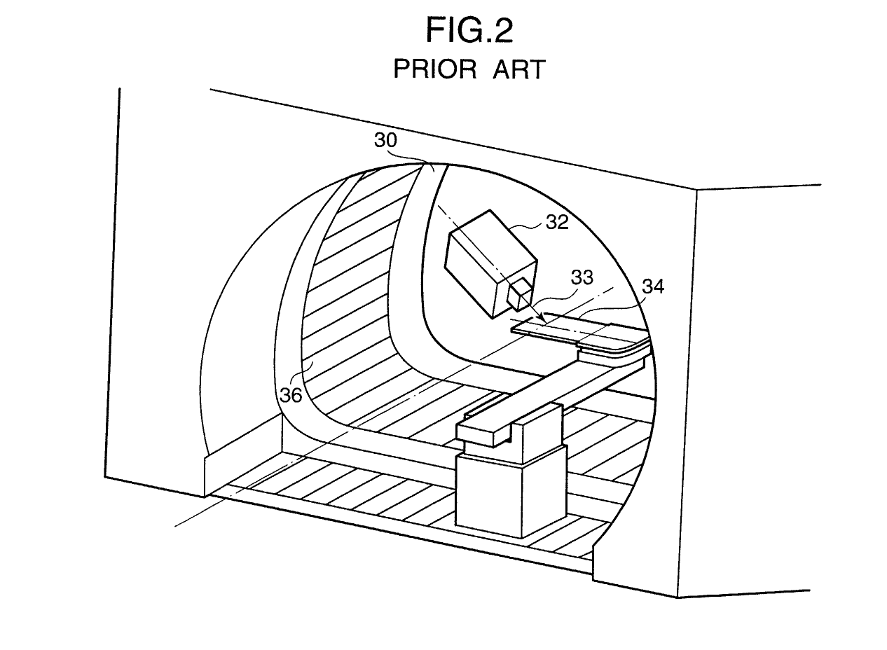

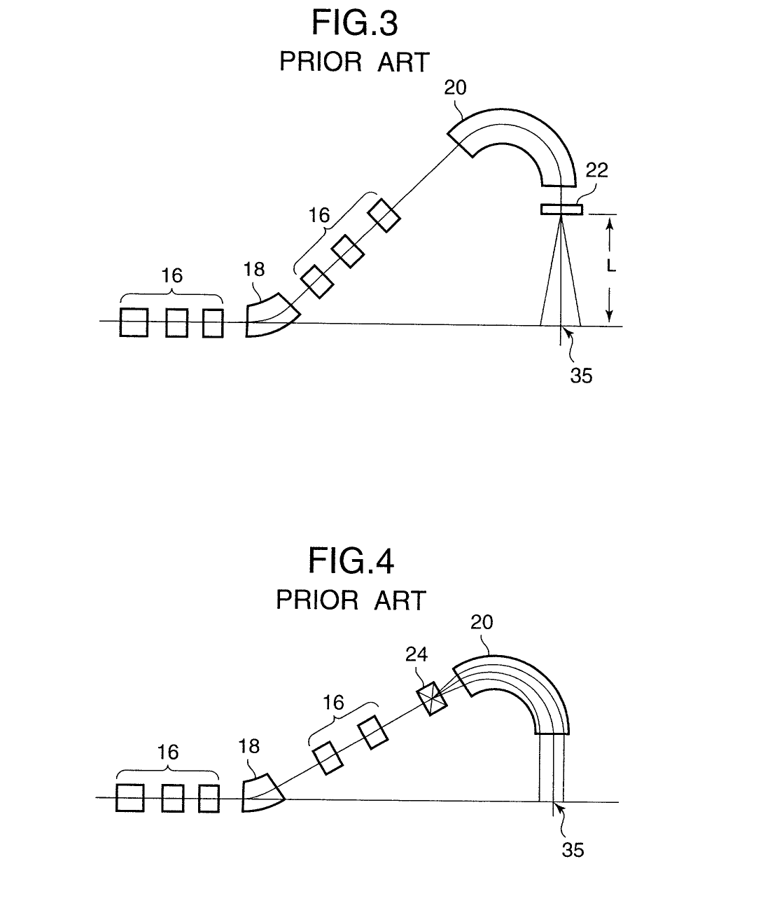

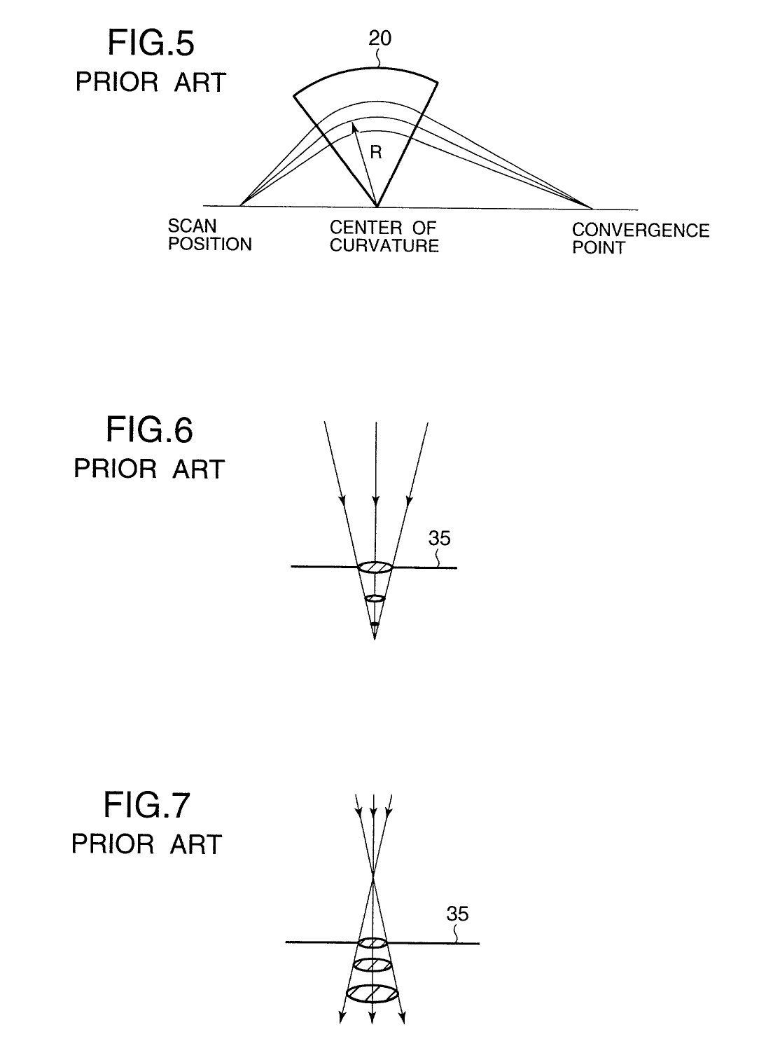

### DETAILED DESCRIPTION OF THE PREFERRED EMBODIMENTS

Preferred embodiments of the present invention will be explained hereunder.

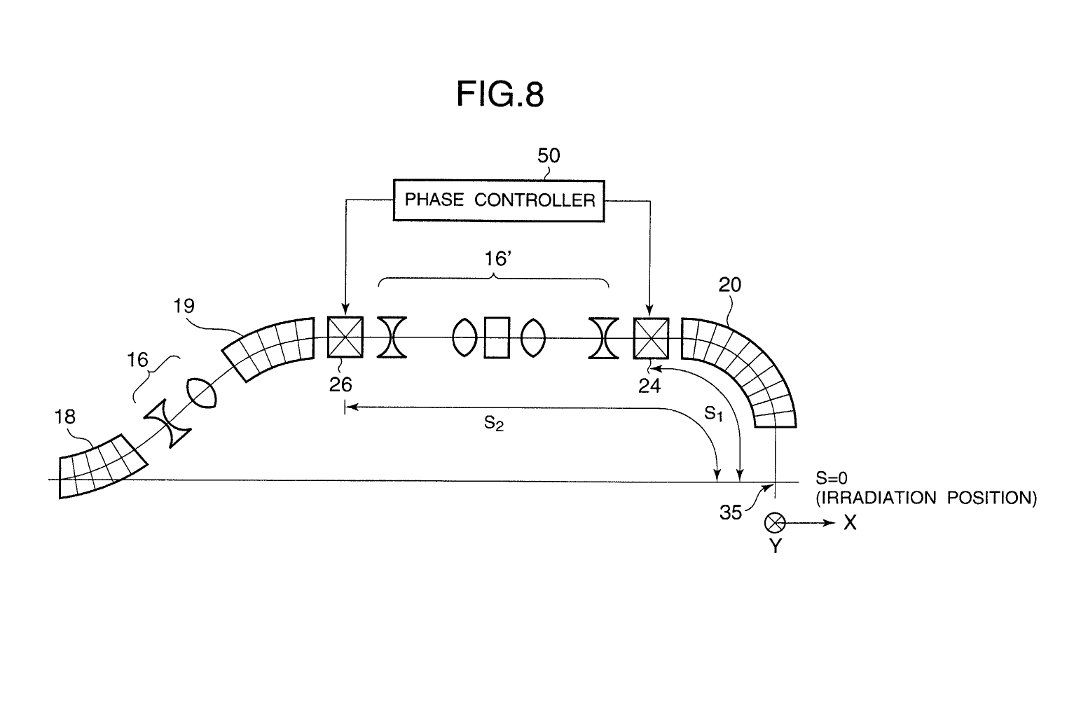

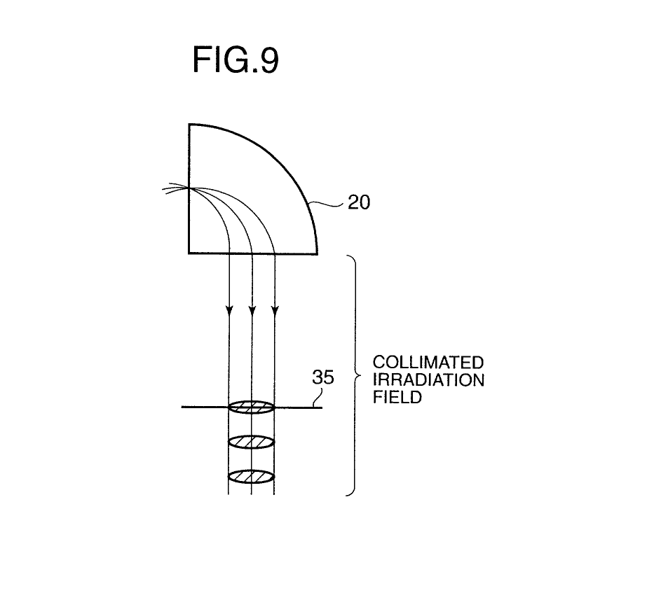

As shown in FIG. 8, in this embodiment, there are interposed two scan electromagnets 24, 26 between a final deflection electromagnet 20 and a deflection electromagnet 19 disposed on an entrance side thereof. Kicks provided by the two scan electromagnets are combined to form a collimated irradiation field, as shown in FIG. 9, on an exit side of the final deflection electromagnet 20. Referring to FIG. 8 is a phase controller 50 synchronously controls the two scan electromagnets 24, 26. Further, two groups of Q magnets 16, 16′ are provided.

Here, the position of the two scan electromagnets 24, 26 is determined as follows.

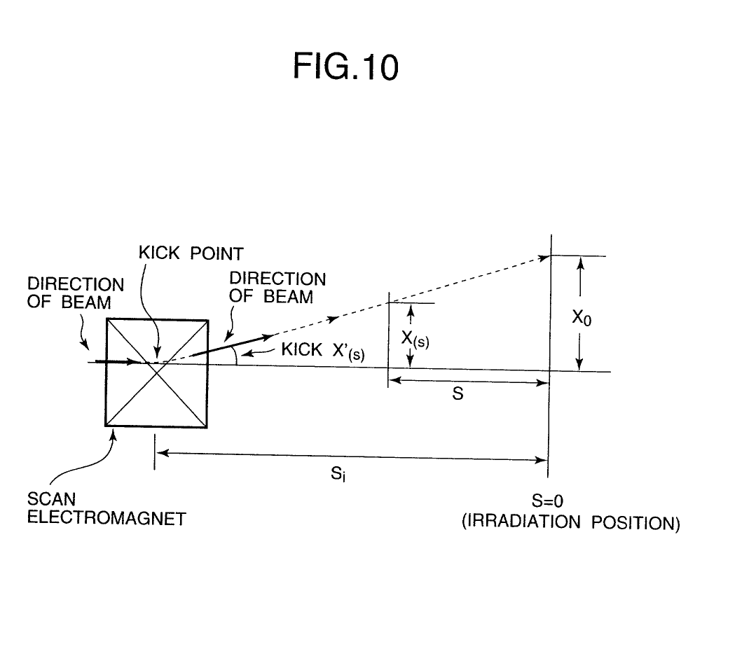

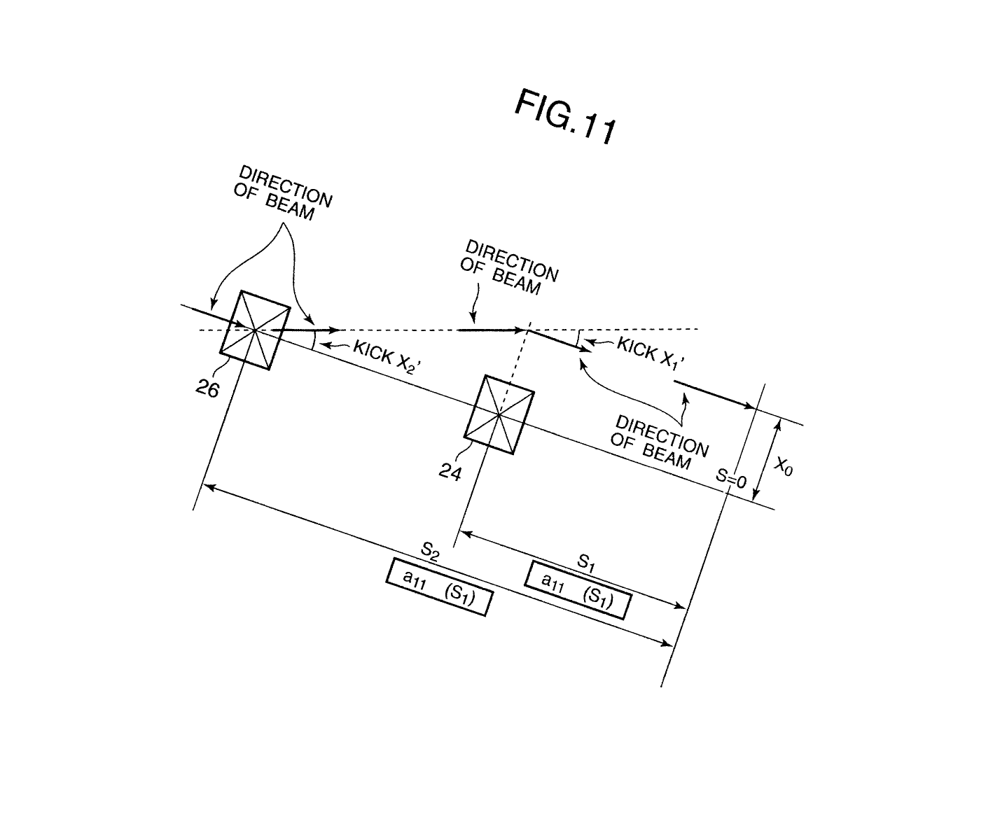

Now, a transport matrix of a beam is inversely determined based on a position (i.e., an irradiation position) of an irradiation portion 35. The displacement X and divergence X′ at distance s from the irradiation position is expressed by the following equation (see FIG. 10). That is,

$$( \begin{matrix}X ( s ) \\ {X}^{′} ( s )\end{matrix} ) = ( \begin{matrix}{a}_{11} ( s ) {a}_{12} ( s ) \\ {a}_{21} ( s ) {a}_{22} ( s )\end{matrix} ) ( \begin{matrix}{X}_{0} \\ {X}_{0}^{′}\end{matrix} ) \qquad ( 1 )$$

where Xo, Xo′ are the displacement and divergence of the beam at the irradiation position (i.e. s=0).

By inversely solving Equation (1), the following equation can be obtained. That is,

$$( \begin{matrix}{X}_{0} \\ {X}_{0}^{′}\end{matrix} ) = ( \begin{matrix}{a}_{22} ( s ) - {a}_{12} ( s ) \\ - {a}_{21} ( s ) {a}_{11} ( s )\end{matrix} ) ( \begin{matrix}X ( s ) \\ {X}^{′} ( s )\end{matrix} ) \qquad ( 2 )$$

Suppose a kick (divergence caused by the scan electromagnet to the beam) is given at point where distance from the irradiation position is s. Then, it holds that X(s)=0 and thon is expressed as follows.

X₀=a₁₂(s)·X′(s) (3)

X₀′=a₁₁(s)·X′(s) (4)

For a beam traveling in the opposite direction, X′(s) becomes −X′(s) and Equations (3), (4) are expressed as follows.

X₀=a₁₂(s)·X′(s) (5)

X₀′=−a₁₁(s)·X′(s) (6)

Therefore, for a given kick at the position of a₁₁(s)=0, it holds that Xₒ′=0 and the beam kicked by the scan electromagnet is necessarily made parallel to the axis downstream from the final deflection electromagnet 20. However, with one scan electromagnet, since the position where a₁₁(s)=0 is immediately adjacent to the entrance of the final deflection electromagnet, no scan electromagnet can be placed there.

In this regard, according to the present invention, a plurality (two in this example) of scan electromagnets are combined to provide the same effect as that of the one scan electromagnet placed at the entrance of the final deflection electromagnet. That is, for the two scan electromagnets 24, 26 placed at points s₁ and s₂, Equations (5) and (6) represent the superposition of two kicks, being expressed as follows.

X₀=a₁₂(s₁)·X₁′+a₁₂(s₂)·X₂′ (7)

X₀′=−a₁₁(s₁)·X₁′−a₁₁(s₂)·X₂′ (8)

In this case, the following equation should be satisfied so that X₀′=0.

a₁₁(s₁)·X₁′+a₁₁(s₂)·X₂′=0 (9)

As shown in FIG. 11, positions s₁ s₂ where each of two electromagnets are placed, (1, 1) component all of the transport matrix thereof, and each kick amount X₁′. X₂′ satisfy relation expressed by equation (9). Therefore, when position s₁ and S₂ are determined, a₁₁(s₁) and a₁₁(s₂) are decided accordingly. Then, when field size X₀ at s=0 is determined, valves of X₁′ and X₂′ are decided by equation (7).

That is, in accordance with a given optical system from point S₂ to the final deflection electromagnet 20, each scan electromagnet would provide the kick determined by Equation (9), thereby making the beam parallel to the axis downstream from the final deflection electromagnet.

This makes it possible to implement a collimated irradiation field at the irradiation position without providing a special shape to the magnetic poles of the final deflection electromagnet.

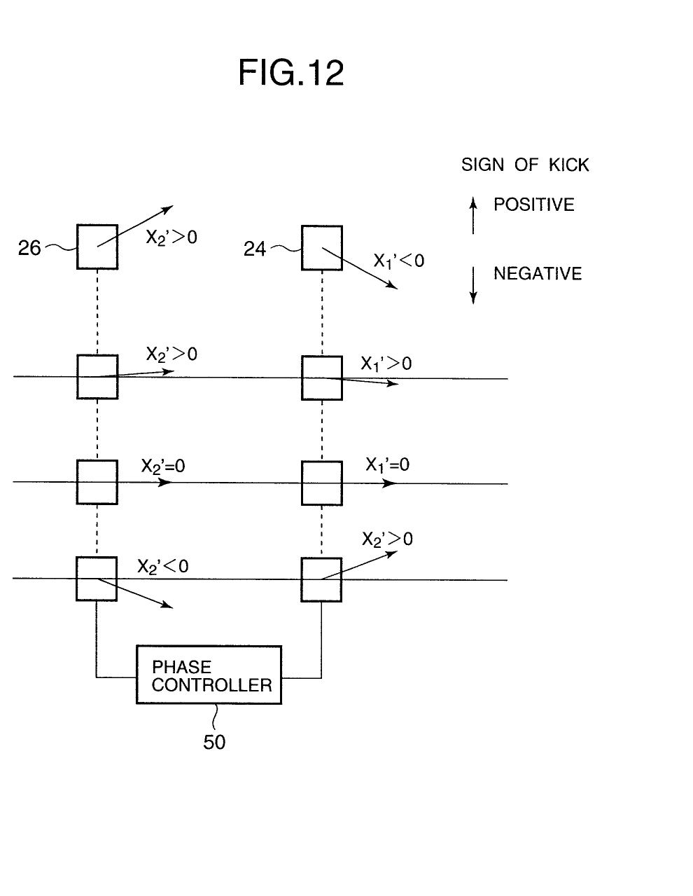

The phase controller 50 controls the two electromagnets 24 and 26 as shown in FIG. 12, if a₁₁(s₁) and a₁₁(s₂) have the same sign. Namely, when kick X₂′ of electromagnet 26 is positive (X₂′>0), then kick X₁′ of electromagnet 24 is negative (X₁′<0). When X₂′=0, then X₁′=0. When X₂′<0, then X₁′>0.

On the other hand, if a₁₁(s₁) and a₁₁(s₂) have different signs, when X₂′>0, then X₁′>0 and when X₂′<0, then X₁′<0.

Incidentally, in the above explanation, two scan electromagnets have been employed, however, the number of scan electromagnets is not limited to two, but may be three or more.

In particular, such scan electromagnets can be arranged that are independent of each other in the X and Y directions, thereby providing collimated irradiation fields in the X and Y directions.

Furthermore, the position of the scan electromagnet is not limited to between the deflection electromagnet 19 and the final deflection electromagnet 20, but maybe upstream from the deflection electromagnet 18.

Furthermore, in the explanation above, the present invention has been applied to the rotary gantry of the proton beam therapy system. However, the present invention is not limited to this application, but may apparently be applied in the same manner to a therapy system or irradiation apparatus, which employs a charged-particle beam other than a proton beam, or to those except for the therapy system.

Although only a limited number of the embodiments of the present invention have been described, it should be understood that the present invention is not limited thereto, and various modifications and variations can be made without departing from the spirit and scope of the invention.

What is claimed is:

1. A charged-particle beam irradiator for allowing a scan electromagnet provided on an entrance side of a final deflection electromagnet to scan a charged-particle beam to expand an irradiation field, said charged-particle beam irradiator, comprising:
a plurality of said scan electromagnets, wherein
kicks provided by the plurality of said scan electromagnets are superimposed to form a collimated irradiation field at an exit of said final deflection electromagnet.

2. A charged-particle beam irradiator according to claim 1, wherein said plurality of scan electromagnets are arranged according to following equation.
a₁₁(s₁)·X₁′+a₁₁(s₂)·X₂′+ . . . +a₁₁(s)·Xₙ′=0
where,
n:number of the electromagnets.
s₁ . . . sₙ : distance from each electromagnet to beam irradiated position
a₁₁(s): coefficient of beam transport matrix
X′: beam divergence at the beam irradiated position

3. A charged-particle beam irradiator according to claim 1 or 2, wherein said plurality of scan electromagnets are interposed between said final deflection electromagnet and a deflection electromagnet disposed on an entrance thereof.

4. A charged-particle beam irradiator according to claim 3, wherein said plurality of scan electromagnets are disposed upstream from said deflection electromagnet at an entrance thereof.

5. A charged-particle beam irradiator according to claim 1 or 2, wherein said plurality of scan electromagnets are disposed independent of each other in X and Y directions.

6. A therapy system, comprising:
a charged-particle beam irradiator, having a plurality of scan electromagnets, configured such that kicks provided by the plurality of said scan electromagnets are superimposed to form a collimated irradiation field at an exit of a final deflection electromagnet to irradiate an affected part with a charged-particle beam.
